(19) United States
(12) Patent Application Publication
(10) Pub. No.: US 2007/0118198 A1
(43) Pub. Date: May 24, 2007

(54) Neurostimulation lead with concentric electrodes

(76) Inventor: Joshua P. Prager, Los Angeles, CA (US)

Correspondence Address: TRASK BRITT, P.O. BOX 2550, SALT LAKE CITY, UT, 84110, US

(21) Appl. No.: 11/593,910

(22) Filed: Nov. 7, 2006

Related U.S. Application Data
(60) Provisional application No. 60/734,624, filed on Nov. 7, 2005.

(51) Int. Cl. A61N 1/05

(52) U.S. Cl. 607/116; 607/117

(57) ABSTRACT

A surgical lead for neurostimulation includes a central cathode circumscribed by an outer anode to define an annular space within which an electromagnetic field is generated between the two electrodes, and associated methods.

### CROSS-REFERENCE TO RELATED APPLICATION

This application claims the benefit under 35 U.S.C. § 119 of U.S. patent application Ser. No. 60/734,624, filed Nov. 7, 2005 the contents of the entirety of which are incorporated by this reference.

### TECHNICAL FIELD

This invention pertains to neurostimulation. It is particularly directed to the leads utilized in neurostimulation procedures, and provides an improved configuration for such leads. This disclosure is particularly directed to leads useful for spinal cord stimulation therapies, but those leads may also find application in brain stimulation therapies.

### BACKGROUND

There are three generally recognized categories of neurostimulation; namely spinal cord stimulation (SCS); brain stimulation, subdivided into deep (DBS) and cortex stimulation, and peripheral nerve stimulation (PNS).

Spinal cord stimulation involves placing a lead in the epidural space posterior to the spinal cord. Electromagnetic fields are induced between electrical contacts carried by the lead, thereby to stimulate specified target areas. The usual target of stimulation is the spinal cord; thus, concurrent stimulation of the nerve root is regarded as undesirable. SCS systems are implantable devices that resemble pacemakers in appearance and function. These systems generate electrical pulses that interfere with the transmission of pain signals to the brain; in effect, painful sensations are replaced by gentle tingling sensations (parethesia). SCS neurostimulation systems have typically included three operably associated components; namely, an implantable power source or receiver, one or more implantable leads and an external controller. The controller may be programmed to customize the electrical pulses delivered through the leads to a particular patient.

Battery powered internal pulse generators and radio frequency transmitter (external)/ receiver (implanted) pulse generators have been alternatively included in SCS systems. Conventional systems currently in use typically include an internal pulse generator (IPG) and one or more leads, current versions of which offer 4, 8 or 16 contacts. A preferred modern SCS configuration includes a fully rechargeable, implantable internal pulse generator capable of delivering electrical pulses in response to multiple simultaneous programs through any of 16 output contacts. This arrangement offers the capability of operating at significantly increased energy levels, because of its inherently decreased need for battery conservation.

The leads of an SCS system may be either percutaneous or surgical. Percutaneous (sometimes called “cylindrical”) leads are emplaced through a hollow needle. In practice, cylindrical leads radiate electricity outward a full 360 degrees. Consequently, less than half of the radiated field is useful. Surgical leads typically carry active contacts on a paddle-shaped, flexible support structure; hence, the common name “paddle lead.” Surgical leads provide a more efficient utilization of power, because they direct the generated field more appropriately for therapeutic applications.

Typically, individual electrodes are embedded within the paddle portion of a surgical lead. Lead wires extend from conductive contact with individual electrodes through a cable extending from the paddle at a position corresponding to the handle of a true paddle. The paddle is shaped and dimensioned as appropriate for surgical implantation. A representative paddle element may, for example, be constructed of medical grade silicone polymer, measuring about 5 centimeters in length, about 1 centimeter in width and about 1/10 centimeter in thickness. The electrodes are embedded in the paddle such that a contact surface of each electrode is exposed at one of the larger surfaces of the paddle.

By convention, electricity is considered to flow from a negative contact (cathode) to a positive contact (anode), thereby creating an electrical field that stimulates the spinal cord. It is known that reducing the spacing between contacts enables deeper penetration of the spinal cord, but with attendant higher energy consumption. Stimulation has traditionally been applied along the spinal cord (longitudinal stimulation), although it can be applied across the cord (transverse stimulation.)

Various electrode patterns have been proposed for surgical leads. For example, one configuration, sometimes referred to as a “linear 4,” positions four solid contacts in a line on a paddle substrate. An alternative surgical lead design, sometimes referred to as a “linear 4×2,” arranges eight active contacts in approximately parallel lines, each including four such contacts.

A “transverse tripole” configuration consists of one or more cathode electrodes flanked by two anodes. This arrangement allows stimulation of the spinal cord without stimulation of the nerve roots, if placed properly. An improved version of this configuration is the “pentad” lead, in which a single cathode contact is positioned within a “linear 2×2” arrangement of anode contacts. The pentad configuration is relatively more effective and forgiving than other linear electrode patterns.

Surgical leads utilizing electrodes are disclosed in U.S. Pat. Nos. 6,909,918, 6,587,733, 6,463,335, 6,360,750, 6,233,488, 6,122,548, and 5,948,007, the contents of the entirety of each of which are incorporated by this reference.

### SUMMARY OF THE INVENTION

In certain embodiments, the present invention provides an improved surgical lead useful for the practice of neurostimulation therapy. The leads of this invention may be used in pain relief and other neurostimulation applications in generally the same fashion as currently available leads are used. They offer several practical and surgical advantages, however. Of central importance to the invention is the arrangement of electrodes that enables the shaping and manipulation of an induced stimulation field.

While other configurations are within contemplation, and may be preferred for specific procedures, this disclosure focuses upon a paddle lead configuration. In contrast to the electrode arrangements previously provided by paddle leads, the electrode pattern of this invention may be viewed as one or more electrode pairs, each of which includes a central cathode enclosed within an anode. The field induced when a voltage drop is applied across the electrode pair is thereby contained within a volume defined by the electrodes.

“Enclosed,” within the context of this disclosure, refers to functional 360-degree containment from a plan view perspective. The “volumes” referred to in this disclosure are defined by the constraints imposed upon a field by the physical presence of the electrodes of the pair. The volume will have an “annular” cross section defined by the electrodes, but may have an indeterminate geometric height (altitude). The effective height of a volume is established by the electrodes, but is also dependent upon other factors influencing the characteristics of the field. From a practical standpoint, however, the field may properly be regarded as “contained” within the annular volume defined by the electrodes.

The term “annular,” is used in this disclosure in a broad sense. That is, the inner cathode need not be circular, or even curvilinear, although for reasons of simplicity, the currently envisioned practical embodiments include cathodes having an approximately circular, oval or other regular geometric cross section. Cathodes of virtually any geometric shape would be expected to be operable and beneficial within the context of this invention, as compared to the electrode patterns of conventional paddle leads. Similarly, the outer anode need not be formed as a circular cylinder, although cylindrical anodes of either circular or oval cross section are currently regarded as most practical from a construction standpoint.

The anode may further be formed of either a continuous or discontinuous wall, which may be perforated, and which need not be of uniform thickness. Without regard to the precise details of its construction, the anode is constructed and arranged to function as a containment shield for the induced field. As a practical matter, the anode, whether integral or in segments, will function to contain the field within a prism-shaped space transverse a plane parallel the anode and cathode electrodes; it will be approximately equivalent in function to a hollow tube formed by a continuous wall. Within the context of this invention, then, the term “concentric” includes an arrangement of a central cathode of any cross sectional shape enclosed within an anode of any hollow configuration. The “annular” space defined by this pair of electrodes may accordingly be irregular in shape.

Contacts may also be slightly curved to conform to the shape of the durra on which the lead will be placed. The concentric electrode pattern of the leads of this invention requires only two conductors, one for the anode and one for the cathode, to accomplish better the performance of the previous “pentode” configuration. Leads may be constructed with any desired number of concentric pairs of electrodes, ranging in number from one to eight such concentric pairs. Up to eight concentric electrode pairs may be coupled to currently available 16 channel (outputs) pulse generators. The concentric electrode pairs of this invention thus provide a significantly enhanced degree of freedom to the treating physician with regard to the design of effective stimulation therapy regimes.

The electrode configuration and arrangement of this invention has the effect of shaping the induced field such that it extends from a reference plane parallel the electrodes within an approximately conical (including frusta-conical) volume. The term “conical” is intended to distinguish from a prism-like shape, in that the cross sectional area of the field is progressively reduced with distance from the reference plane. It should be understood that a “conical” volume, within the context of this disclosure, need not be characterized by circular cross sections, and is permitted to be irregular. In any case, the field induced between the electrodes of this invention permits deeper and more precise therapeutic application than has heretofore been possible in SCS and brain stimulation procedures.

### BRIEF DESCRIPTION OF THE DRAWINGS

In the drawings, which illustrate what is currently regarded as the best mode for carrying out the invention:

FIG. 1 is a plan view of a surgical lead of this invention;

FIG. 2 is a fragmentary view in elevation of the portion of FIG. 1 within the boundary line 2-2, drawn to an enlarged scale;

FIG. 3 is a fragmentary plan view of the portion of FIG.1 within the boundary line 3-3, drawn to an enlarged scale;

FIG. 4 is a view in section of the components of FIG. 3, taken along the section line 4 4; looking in the direction of the arrows; and

FIGS. 5 through 10 are fragmentary plan views similar to FIG. 3, but illustrating alternative embodiments.

### DETAILED DESCRIPTION OF THE INVENTION

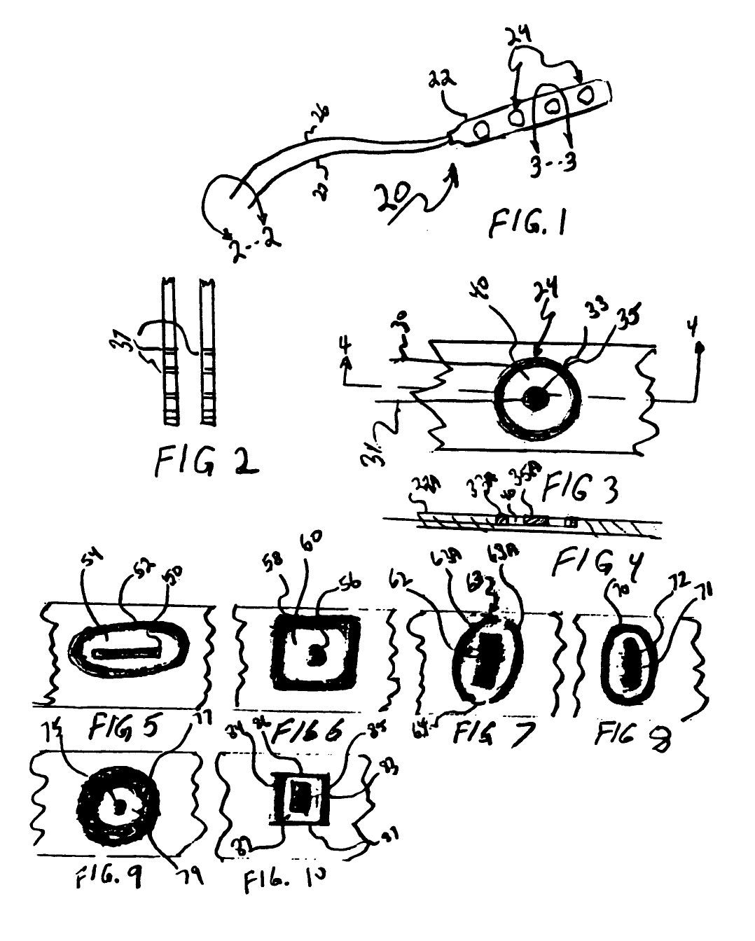

The surgical lead, generally 20, shown by FIG. 1 is constructed in accordance with current technology, and comprises a paddle-shaped flexible mounting base 22, carrying four electrode pairs, generally 24, of this invention. Two conductor cables 26, 27 extend from the base, and house fine wires 30, 31 (FIG. 3), which are conductively connected in conventional fashion to the anode 33 and cathode 35, respectively, of an electrode pair 24. As best shown by FIG. 2, each of the cables 26, 27, includes four wire conductors (e.g., 30, 31, FIG. 3), each individually terminating in a cylindrical electrical contact 37, adapted for plug-in connection to the output channels of a pulse generator in typical fashion. The lead of FIG. 1 is constructed with four electrode pairs 24, which require only half (eight) of the output contacts available in a 16 channel pulse generator to operate. Applying a voltage (conventionally in the millivolt range) between the electrodes 33, 35 induces a field within the annular region 40. Referring to FIG. 4, the electrodes 33, 35 are embedded in the flexible mounting base (“paddle”) 22 with contact surfaces 33A, 35A projecting slightly through an “active” surface 22A of the paddle 22.

The various embodiments illustrated by the drawings are illustrative only, and the respective figures are schematic in nature. It is recognized that both the geometries and electromagnetic properties of the fields induced by the leads of this invention are dependent, in predictable fashion, upon a number of variables, any of which may be selectively specified to achieve a desired therapeutic or operational consequence. Among those variables are the specific and relative sizes and shapes of the anode and cathode components of each electrode pair, the materials of construction of the electrodes, the specific and relative potentials applied to the anode and cathode, respectively, the size and shape of the annular volume between the electrodes, and the porosity of the anode.

FIGS. 5 through 10 illustrate the variability of patterns of electrode pairs available to the practitioner in accordance with this invention. It is within contemplation that a plurality of such electrode pairs be carried by a single paddle structure, and that more than one pattern be included in a single lead. FIG. 5 illustrates a pattern in which an elongate rectilinear cathode 50 is circumscribed by an ovoid anode 52, thereby defining a somewhat irregular annular volume 54. The cross sectional area of the volume 54 may be reduced or enlarged by altering the dimensions of either the cathode 50 or anode 52. FIG. 6 illustrates an alternative pattern in which relatively small diameter circular cathode 56 is positioned within an approximately rectilinear anode 58. Again, the cross sectional area of the annular volume 60 is dependent upon the relative sizes selected for the cathode 56 and anode 58. FIG. 7 illustrates a rectilinear cathode 62 of relatively large cross sectional area between two halves 63A, 63B of an anode, generally 63, which are conductively connected by a wire 64. Accordingly, the anode 63 functions electrically approximately as though it were integral, similar to the anode 70 shown by FIG. 8. The annular volume 71 defined by the anode 70 and cathode 72 is approximately oval-concentric.

The cathode 75 illustrated by FIG. 9 is relatively smaller in diameter than the corresponding element 35 illustrated by FIG. 3. Conversely, the anode77 of FIG. 9 has relatively larger cross sectional area than the anode 33 of FIG. 3, resulting in a relatively smaller annular volume 79. FIG. 10 illustrates an electrode pair that defines an approximately rectilinear annular volume 82 by positioning a rectilinear cathode 83 approximately parallel and between relatively thick conductive components 84, 85, physically and conductively connected by relatively thin conductive components 86, 87. The components 84, 85, 86 and 87 together comprise a single anode, within the context of this disclosure.

What is claimed is:

1. A surgical lead comprising:
a pair of electrodes operable to generate an electromagnetic field between them, including a central cathode circumscribed by an outer anode spaced from said cathode, said electrodes being structured and arranged to confine said field within an approximately annular space defined by said electrodes.

2. The surgical lead of claim 1, wherein said anode is curvilinear in cross section.

3. The surgical lead of claim 2, wherein said cathode is curvilinear in cross section.

4. The surgical lead of claim 2, wherein said anode is constructed as a continuous wall defining a hollow interior space, open at opposite ends.

5. The surgical lead of claim 4, wherein said cathode is approximately centrally disposed within said anode, whereby to define a geometrically regular annular space.

6. The surgical lead of claim 1, said surgical lead comprising a plurality of such pairs of electrodes.

7. In a surgical lead of the type that comprises a paddle-shaped base support carrying a pair of spaced electrodes including a cathode and an anode, the improvement comprising:
said anode being spaced from said cathode and being shaped and dimensioned to surround substantially the entire perimeter of said cathode about an axis transverse a reference plane transverse both said cathode and said anode.

8. The improvement according to claim 10, wherein said cathode and said anode together define an approximately annular space.

9. A method of performing neurostimulation in a subject, said method comprising:
applying the surgical lead of claim 1 to the subject.
